(19) United States
(12) Patent Application Publication
(10) Pub. No.: US 2020/0061238 A1
(43) Pub. Date: Feb. 27, 2020

(54) Ceramic Sliding Bearing

(71) Applicant: CeramTec GmbH, Plochingen (DE)

(72) Inventors: Mateusz Maria JUSZCZYK, Velden (DE); Michael GÖTZ, Uttenreuth (DE)

(21) Appl. No.: 16/498,552

(22) PCT Filed: Mar. 22, 2018
(86) PCT No.: PCT/EP2018/057327

§ 371 Date: Sep. 27, 2019

(30) Foreign Application Priority Data
Mar. 28, 2017 (DE) 10 2017 205 173.6

(51) Int. Cl. A61L 27/10; A61L 27/56; A61L 27/42; A61F 2/32; A61F 2/38; A61F 2/40; A61F 2/42; C04B 35/48; C04B 35/10; C04B 35/584; C04B 35/565; C10M 103/06; F16C 33/04

(52) U.S. Cl. CPC A61L 27/10; A61F 2002/307; A61L 27/105; A61L 27/427; A61F 2/32; A61F 2/38; A61F 2/40; A61F 2/42; C04B 35/48; C04B 35/10; C04B 35/584; C04B 35/565; C10M 103/06; F16C 33/043; A61F 2310/00203; A61F 2310/00281; A61F 2310/00239; A61F 2310/00317; A61L 27/56

(57) ABSTRACT

Disclosed is a ceramic sliding partner for a sliding bearing, said sliding partner being made at least in part, preferably entirely, of a ceramic foam. The ceramic sliding partner comprises at least one sliding surface on which a sliding partner can move, said sliding surface being made at least in part, preferably entirely, of a ceramic foam.

The invention relates to the use of a porous ceramic part as a sliding partner A in conjunction with a preferably ceramic sliding partner B, which moves against the sliding partner A. The invention preferably relates to the use of the sliding partners in the field of medical technology.

Sintered bearings are known from the prior art as a type of sliding bearing, in which the bearing shell consists of a sintered, porous metal. The pores are filled with lubricants, such as oils. Due to the porosity, large quantities of lubricants can be stored. Bearings made of sintered metals thus represent at least partly self-lubricating bearings. Sintered bronze or sintered iron are used as sintered metals, for example.

In sintered air bearings, the porous material of the sliding bearing ensures an even distribution of the air. The advantage of this is quiet running and low wear. However, bearings of this kind have high dead volumes. Since the porosity is not evenly distributed over the material, the air flows out of the bearing unevenly. The focus with porous ceramic bearings that are used at present is on high-temperature applications, dry-run applications or applications with high accuracy requirements in respect of positioning.

JP2008150233 discloses a porous sliding partner made of a ceramic material, into the pores of which a lubricating or sliding agent can be introduced. The ceramic material consists of zirconium oxide in this case.

Durazo-Cardenas et al. (Proc. IMechE Vol. 224 Part J: J. Engineering Tribology (2010) p. 81-89) demonstrate a hydrostatic sliding bearing made of a ceramic material produced by means of a starch consolidation (SC) method. For this purpose, aluminum oxide powder is mixed with starch granules and water and cast into shape. At temperatures around 70° C., the starch swells under water absorption and the mass is thus solidified. Under sintering conditions, the starch grains burn and leave pores in the ceramic material. In comparison with a similar sliding bearing made of sintered iron, a higher static stiffness and a higher torsional stiffness could be achieved. As a result, the hydrostatic pressure is better distributed and hydrodynamic effects can be improved.

In general, a variety of methods and processes are known for producing the porous structures. These include, for example, slip-based methods in which, by means of a ceramic slip having organic, structure-determining porosity-enhancing agents or chemical ingredients, porous ceramic structures are produced on components or thoroughly porous components are produced. The ceramic slips are to be understood as suspensions comprising a liquid medium, a ceramic starting powder and optionally additional additives.

The problem with conventionally produced ceramic parts which have or consist of a porous part is that the metal-free porous structures often have only low stabilities and are difficult to process intraoperatively, for example. The introduction of screws or nails, for example for temporarily securing the ceramic part, can lead to a catastrophic failure of the porous structure or of the entire implant in the case of porous structures produced by known methods.

The sliding bearings from the prior art having a hard-on-hard coupling exhibit excellent abrasion resistance. However, there is noise when the geometry of the sliding bearing together with the modulus of elasticity generates and amplifies audible frequencies.

The object of the present invention was therefore that of providing ceramic sliding bearings consisting of at least two sliding partners, which do not have the disadvantages of the prior art and significantly reduce noise, preferably completely avoiding it.

The invention is achieved by the sliding partner A according to claim 1. The sliding partner A consists at least partly, preferably completely, of a ceramic foam and has at least one sliding surface, which is at least partly formed from the foamed ceramic material. A sliding partner B is intended to move against this sliding surface. Preferred embodiments are specified in the dependent claims.

The sliding bearing consists of a sliding partner B, which moves against the porous sliding partner A, and the sliding partner A. In one embodiment, the two sliding partners are made from ceramic material. In a preferred embodiment, the sliding partner B is made from solid ceramic material. In an alternative embodiment, the sliding partner B is also at least partly porous, and is preferably also at least partly made from a ceramic foam.

Foamed ceramic parts within the meaning of the present invention are parts made of ceramic material, which consist partly, preferably completely, of a ceramic foam. The ceramic foam consists of bulk ceramic material which has a significant proportion of pores (usually 20 to 95% by volume) which may be isolated (closed porosity) and/or in a pore network (open porosity).

The term ceramic material refers to inorganic, non-metallic sintered materials. The ceramic material is preferably selected from sintered metal oxides, carbides or nitrides.

Porous means a foamy or spongy, porous material, which has holes, in which pores may contain air. Porous substances are heterogeneous due to the air/liquid contained in the pores. Permeable refers to a substance that is permeable to certain substances.

The sliding bearing consists of at least two parts, the sliding partners A and B, which are arranged so as to be movable relative to one another. In this case, one of the two sliding partners, for example, the sliding partner A, can be stationary, and the sliding partner B can be arranged so as to be movable relative to the sliding partner A. It is also possible for the sliding partner B to be stationary and the sliding partner A to be movably arranged. In a particular embodiment, both sliding partners A and B are movably arranged and can move relative to one other. The movement of the two sliding partners can be a rubbing movement. According to the invention, the sliding partner B is at least partly made of a polished, ceramic material, and preferably is at least partly solid, such that the sliding surface thereof is designed to be solid, i.e. having a proportion of <10% pores, preferably <5% pores, particularly preferably without pores, based on the total surface area, and the sliding partner is particularly preferably fully solid. The sliding partner A is at least partly made from a ceramic foam. The sliding surface thereof is at least partly arranged in the region consisting of the foamed ceramic material. It thus has at least one sliding surface, which consists of a ceramic foam and has a porous surface.

The following are examples of porous ceramic parts that have different ceramic structures in which the ceramic foam has different characteristics:

Full foam part: A part of which 100% of the volume consists of ceramic foam. It can be used in medical technology for example as a sliding shell which is operatively connected to a spherical sliding partner B, preferably made of ceramic material, in order to be used for osteoconduction and osseointegration on account of its property as a guide structure. Ceramic foam is used as the basis for the sliding surface on which the sliding partner B moves. This sliding surface can also be processed for finishing. The sliding surface is made from the ceramic foam.

3D-structured part: A part that consists of both a porous region and a significant, dense, ceramic region. The porous region usually protrudes into the part by more than 1 mm. Examples thereof are partial resurfacing implants in which the bone-facing region of the part is extensively porous, and a region of the part comprises a dense ceramic region. In this case, the articulation surface on which the sliding partner B moves consists at least partly, preferably completely, of the porous ceramic part.

2D-textured part: A part of which the surface topology is partly or completely defined by means of a thin, near-surface, porous region. The porous region projects approximately ≤1 mm deep into the part, meaning the volume fraction of the dense, ceramic material is greater than in the 3D-structured part. Examples thereof are ceramic monobloc ball sockets in which the pelvis-facing rear side is open-pored and textured, and the side facing the hip joint ball at consists at least partly of the porous ceramic material and is optionally partly formed from dense, polished, preferably ceramic material.

According to the invention, parts of which the cross sections are formed from different structures are possible. These structures may include both porous ceramic foam, as well as dense ceramic materials, with the arrangement of the structures being determined by the application of the parts. As a result, any desired combinations of the above structures are conceivable.

In a preferred embodiment, the ceramic sliding bearings, of which sliding partner A has a sliding surface which consists at least partly of a ceramic foam, are ceramic implants, i.e. can be used in both human medicine and in veterinary medicine as implants for small animals, farm animals and pets, and particularly preferably are implants for human medical applications.

Implants preferred according to the invention, which usually have wall thicknesses in the range of from 0.3 to 30 mm, for human medical applications are implants for small and large joints, implants in the field of partial resurfacing, as well as components or parts of implant systems.

Implants according to the invention for small joints may in particular comprise implants for the finger joints, toe joints, elbow joints, ankle joints and the wrist and other joints. The term implants for large joints includes for example implants for the hip joint, the knee joint and the shoulder joint.

The term partial resurfacing in the context of the present invention includes partial prostheses that compensate only for local joint/cartilage defects. Such prostheses usually consist of a tribologically optimized, congruent side, which faces the joint cavity, as well as a side facing the bone, which ensures the anchoring. Partial resurfacing is mainly used for large joints, since less (bone) tissue has to be removed due to the smaller overall operating area and, as a result, it is considerably easier to carry out subsequent revision operations.

Ceramic parts consisting of structures according to the invention can also be used as components in implant systems. In this case, the porous region, when it is used facing the bone, facilitates osseointegration.

In a further embodiment, the sliding bearing is used as a technical sliding bearing in a joint or a linear, radial, axial and/or radiax bearing. Due to its properties, it is used in applications where extremely high demands, such as speeds, are set, e.g. in turbine wheels.

In this case, the side of the ceramic foam facing the sliding surface is preferably provided with a sliding agent, also referred to as lubricating agent, solid lubricating agent and/or sliding bearing materials.

Sliding agents are selected in one embodiment from liquids, preferably oils or water or mixtures thereof. In a further embodiment, the sliding agents are selected from solid lubricating agents, preferably, graphite, MoS2 or hexagonal boron nitride. In an alternative embodiment, the sliding agents are selected from suspensions, preferably copper or MoS2 in oils. The sliding agents reduce the friction coefficient of the sliding bearing. The sliding agents in the form of liquids or suspensions of liquids and solids can be replenished during the articulation.

In a further embodiment, sliding bearing materials are used instead of a sliding agent or in addition to the sliding agent. The sliding bearing materials provide lubrication in the case of a dry run and therefore have the same function as the sliding agent. The sliding bearing materials are preferably selected from polytetrafluoroethane (PTFE), polyethylene (PE), polyvinyl chloride (PVC), bronze, brass or aluminum alloys. The sliding bearing materials are self-lubricating solids that are not replenished.

In a preferred embodiment, the plastics materials, such as PE and PVC, are applied to the articulation surface by plastic infiltration.

In one embodiment, the sliding agent or the sliding bearing materials are introduced into the bearing cavity and/or the preferably open porosity before assembly. In a further embodiment, the sliding agent is supplied during operation, i.e. conveyed preferably by pressure and/or capillary forces from outside to inside. The sliding agent is supplied and/or distributed in another embodiment by hydrodynamic pressure.

In a particular embodiment, the sliding agent corresponds to the surrounding medium, such as oil, water and/or body fluids, such as in the case of a medical application in which the ceramic part is used as an implant.

On account of the porous region of a structure according to the invention, the connection to other non-ceramic materials is also possible or improved. This makes it possible to bond structures according to the invention to other materials, for example by plastic infiltration or gluing. Ceramic and non-ceramic structures may be connected, with the porous region of the ceramic structure making it possible to have a firm, preferably permanent connection to the non-ceramic material. This can involve an integrally bonded connection of two parts. This integrally bonded connection can be produced exclusively by a thermal process. It can also be produced by an additional material, such as an adhesive. It is also possible for two parts to be connected by a combination of a thermal process and additional material.

The macrostructure of the porous region of the sliding partner A is dominated by the pores, the pore size of the porous region of the part being at least 1 nm, preferably 10 μm, particularly preferably 50 μm and more particularly preferably 100 μm. The maximum size of the pores is 1 mm, preferably 700 μm. The pore sizes are determined by means of microscope images having a resolution of at least 0.2 pixels/pm and preferably having a resolution in the range of from 0.2 to 1 pixels/μm by software-assisted marking and subsequent calculation of the equivalent diameter. By suitably selecting the pore size, the biological, in particular osseointegrative, properties can be significantly improved.

The pores are spherical and/or elongate and/or irregularly shaped. The pores are monomodal in a preferred embodiment, and multimodal in a further embodiment. In one embodiment, the pores are distributed homogeneously over the entire sliding partner. In another embodiment, the pores are distributed in a graduated and/or hierarchical manner, i.e. there are local gradients or gradients extending over the entire part.

The porous region also preferably has a porosity of from 20 to 95%, preferably 55 to 85%. In contrast, the optionally present dense or solid region has a residual porosity of at most 10%, preferably at most 5%, particularly preferably having no residual porosity.

In the case of 3D-structured parts, the porosity is preferably predominantly open porosity, which forms an interconnecting pore network, with at least 60%, particularly preferably at least 85%, of the porosity constituting open porosities. The interconnecting pore network having the above-mentioned pore sizes makes it possible for the osseointegration to also go beyond the near-surface, truncated pores, into deeper pores. The ingrowth can take place to depths of more than 0.5 mm, up to 5 mm. At the same time, on account of the deeper ingrowth, mechanical interlocking of the implant and the surrounding tissue or bone can be achieved by undercut pores.

In addition, the open porosity allows nutrients to be supplied by a diffusion processes in the extracellular fluid. Moreover, micromechanical strains and thus hydrodynamic circulation processes can occur in the porous region of the part, in particular of the implant according to the invention, which has a reduced modulus of elasticity under mechanical stress. The modulus of elasticity of the ceramic foam is approximately ≤15%, preferably ≤10%, of the modulus of elasticity of the bulk ceramic material.

These properties of the ceramic parts, in particular of the implants, can be realized very well by means of foaming methods in which defined pore structures are produced in principle on the basis of foaming or blowing agents in a ceramic slip.

The use of a foaming method is also advantageous in that, in comparison with known forms of ceramic slip preparation, it can be implemented without significant additional effort when there is proper process control. For example, no additional shaping structures are required, such as organic balls of cellulose, fiber structures or polyurethane foam structures that are soaked in specially prepared ceramic slips and then have to be burned out later on in the manufacturing process (porosity method, template burnout or conversion, etc.).

The ceramic material for the sliding partner A according to the invention and/or the sliding bearing can be made from known and commercially available (ceramic) materials. When using the ceramic sliding bearing as an implant, the selection of materials for the two sliding partners is made with the proviso that the ceramic material is biocompatible and preferably exhibits higher strengths, lower corrosion behavior and lower ion-release rates in the body than calcium phosphates such as hydroxyapatite (HA) and tricalcium phosphate (TCP) or metals and alloys.

The optionally present at least two regions of the ceramic part, i.e. at least one porous region consisting of the ceramic foam and at least one dense region, may consist of the same or a different ceramic material.

Preferred ceramic materials, thus including the starting powders for producing the sliding partner A and/or B according to the invention, are oxide-ceramic materials, for example based on aluminum oxide or zirconium oxide, or non-oxide ceramic materials, based for example on silicon nitride or silicon carbide. The basic requirement for the material when used as an implant is the biocompatibility thereof, i.e. it must not cause negative reactions in the body. In this particular case, the product has to satisfy the biological evaluation e.g. according to DIN EN ISO 10993 (as of 2010-04).

In a preferred embodiment, the ceramic material is a material consisting of the mixed oxide system Al2O3-ZrO2, in particular ZTA ceramic materials (zirconia toughened alumina), or ceramic composite materials in which zirconium oxide represents the volume-dominating phase, with chemical stabilizers or dispersoids in the form of further metal oxides or mixed oxides also being added to said systems depending on the dominating phase. Toxic materials can also be used for technical sliding bearings.

Examples of ZTA ceramic materials in which aluminum oxide is the volume-dominating phase are:
A ceramic material consisting of 60 to 98 vol. % of an aluminum oxide/chromium oxide mixed crystal as a matrix material, which can contain 0.8 to 32.9 vol. % of one or more further mixed crystals selected from mixed crystals according to one of general formulas La0,9Al11,76-xCrxO19, Me1Al11-xCrxO17, Me2Al12-xCrxO19, Me2′Al12-xCrxO19 or Me3Al11-xCrxO18, where Me1 is an alkali metal, Me2 is an alkaline earth metal, Me2′ is cadmium, lead or mercury and Me3 is a rare earth oxide metal, and where x corresponds to a value of from 0.0007 to 0.045, and consisting of 2 to 40 vol. % of zirconium dioxide embedded in the matrix material, which can contain, as stabilizing oxides, more than 10 to 15 mol. % of one or more of the oxides of cerium, praseodymium and terbium and/or 0.2 to 3.5 mol. % yttrium oxide, based on the mixture of zirconium dioxide and stabilizing oxides. A ceramic material consisting of aluminum oxide as a ceramic matrix with zirconium oxide dispersed therein and optionally further additives or phases, the aluminum oxide content being at least 65 vol. % and the zirconium oxide content being from 10 to 35 vol. %, the zirconium oxide, based on the total zirconium oxide content, being present in the tetragonal phase by 80 to 99%, preferably by 90 to 99%, and the tetragonal phase of the zirconium oxide being largely mechanically rather than chemically stabilized, the total content of chemical stabilizers being <0.2 mol. %, with preferably no chemical stabilizers being used. This material preferably contains a further dispersoid phase, the volume fraction of the dispersoids forming the dispersoid phase being up to 10 vol. %, preferably 2 to 8 vol. %, particularly preferably 3 to 6 vol. %. According to the invention, in principle all substances which are chemically stable and which, during the production of the composite material, do not dissolve in the aluminum oxide or in the zirconium oxide by sintering at high temperatures and, due to their crystal structure, allow inelastic microdeformations on a microscopic level, can be used as dispersoids. According to the invention, both the addition of dispersoids and the in-situ formation of the dispersoids during the production of the composite material according to the invention are possible. Examples of dispersoids that are suitable according to the invention are strontium aluminate (SrAl12O19) or lanthanum aluminate (LaAl11O18).

An example of ceramic composite materials in which zirconium oxide is the volume-dominating phase is a ceramic material with a ceramic matrix consisting of zirconium oxide and at least one secondary phase dispersed therein, the matrix consisting of zirconium oxide accounting for at least 51 vol. % of the composite material, and the secondary phase accounting for 1 to 49 vol. % of the composite material, the zirconium oxide, based on the total zirconium oxide content, being present in the tetragonal phase by 90 to 99%, preferably 95 to 99%, and Y2O3, CeO2, Gd2O3, Sm2O3 and/or Er2O3 being contained as chemical stabilizers, the total content of chemical stabilizers being <12 mol. % based on the zirconium oxide content, and the secondary phase being selected from one or more of the following compounds: Strontium hexaaluminate aluminate (SrAl12O19), lanthanum aluminate (LaAl11O18), hydroxyapatite (Ca10(PO4)6(OH) 2), fluorapatite (Ca10(PO4)6F2), tricalcium phosphate (Ca3(PO4)2), spinel (MgAl2O4), aluminum oxide (Al2O3), yttrium aluminum garnet (Y3Al5O12), mullite (Al6Si2O13), zircon (ZrSiO4), quartz (SiO2), talc (Mg3Si4O10(OH)2), kaolinite (Al2Si2O5(OH)4), pyrophyllite (Al2Si4O10(OH)2), potassium feldspar (KAlSi3O8), leucite (KAlSi2O6), and lithium metasilicate (Li2SiO3); strontium hexaaluminate, lanthanum aluminate, hydroxyapatite, fluorapatite, spinel, aluminum oxide and zirconium being preferred, and strontium hexaaluminate being particularly preferred.

The average particle size (D50) of the ceramic starting powder can be determined by laser diffraction and, according to the invention, is preferably in the range of from 0.01 to 50 μm, particularly preferably in the range of from 0.1 to 5 μm.

Usually, the grain size in the sintered structure is in a similar range of from 0.01 to 50 μm or particularly preferably in the range of from 0.1 to 5 microns, determined in the microstructure by means of line intersection methods according to DIN EN ISO 13383-1 (2016-11).

The ceramic part according to the invention consists in one embodiment at least of a porous region and optionally a dense region, the porous region, which consists of a ceramic foam, preferably having a density in the range of from 0.5 to 2.5 g/cm³, particularly preferably 0.8 to 1.8 g/cm³. The strength of the porous region of the part is preferably in the range of from 5 to 300 MPa, particularly preferably in the range of from 20 to 150 MPa.

The thermal conductivity of the ceramic part is preferably <10 W/Km and thus lies in a similar range to the thermal conductivity of the natural tissue.

As a result, an altered cold/hot sensation is reduced for the user or patient, preferably completely prevented, by the use of an implant.

By using a structure according to the invention comprising a ceramic foam, the behavior of this structure is significantly altered. In the case of high local loads, especially under pressure, there is thus a locally limited defect instead of a catastrophic failure of the entire ceramic part. The local damage manifests itself as fractures in the pore webs and is limited to the region comprising the porous foam. The cracks are prevented from spreading further, since this material has a low fracture toughness (<1 MPam^{1/2}). It comprises pores that continually counteract the spread of cracks with new interfaces. This locally restricted material behavior leads to compacting of the material of the porous region, it being possible for deformation energy to be dissipated and, in addition, for applied voltages to be distributed and thereby diminished.

This material behavior of a part according to the invention allows machining methods such as drilling, nailing, screwing, rasping, and abrasive cutting. This makes it possible to secure a part according to the invention by fastening means such as screws, nails, pins, etc. These fastening means can be introduced into the region formed by the porous ceramic foam without the part being damaged, which impairs the use.

This has the consequence that, when used as an implant, the part according the invention, in particular the porous region consisting of the ceramic foam, not only promotes the ingrowth of the natural tissue, but also contributes to the securing before and during the operation, i.e. a connection to the body or other implant material is made possible. The ceramic part of the present invention, or the porous region thereof, can preferably be screwed, i.e. screws can be introduced, nailed, i.e. hammering or pressing-in of nails is made possible, and drilled, i.e. holes can be made, as a result of which further form-fitting and/or frictional connections (e.g. by pins), as well as stitching, are also made possible. Said fixing means may have a diameter of up to 5 mm, preferably up to 3 mm.

The ceramic part or the porous region thereof, which does not include the sliding surface, can also be glued and welded (Bone Welding®). Both in gluing and in Bone Welding®, the porosity of the part according to the invention or of the porous region thereof is advantageous, since the implant can be infiltrated with the process material (>0.5 mm deep) and then, going beyond a chemical bond, can also be mechanically connected thereto, for example interlocked therewith. As a result, connections to other materials, for example non-ceramic materials such as plastics and metals, are possible. The different joining methods of the different materials can be carried out within applications, for example when used during an operation, or separately therefrom, in advance when manufacturing a component or part of a system.

Sliding partners obtained in this way have the following advantages:
Pores are used as a sliding agent reservoir.
The ceramic-specific wetting behavior is improved because the pores lead to a rough surface.
Pores can eliminate and/or absorb the abraded material and/or introduced dirt.
The porous sliding partner has a lower stiffness due to the porosity. Mechanical damping can thus take place.
High abrasion resistance, which can be adapted to the sliding partner.
By virtue of the porosity, the sliding bearing has a lower density and is thus suitable for use in lightweight construction applications.
In the case of a sudden load which separates the sliding partners, the permeable (open) pore structure reduces the adhesion force between the two sliding partners.
Good damping behavior under vibration (e.g. to avoid squeaking of the bearing or to dampen imbalances)
Due to the porosity, the sliding bearings have the ability to carry loads with and without lubricant. They exhibit very good dry-run properties, but can also be operated using a variety of above-mentioned lubricants.
Broad temperature range of from −200 to 2,000° C.
Very good thermal shock resistance
Thermal insulation behavior
Corrosion resistance e.g. for use in chemically aggressive environments
Bioinert behavior on account of the ceramic material
Low radial deviation during axis movement
High mechanical load-bearing capacity
Uniform pressure distribution of hydrostatically introduced lubricants

1. Ceramic sliding partner for a sliding bearing, which partner consists at least partly of a ceramic foam, the ceramic sliding partner comprising at least one sliding surface on which a sliding partner is movable, wherein the sliding surface consists at least partly of a ceramic foam.

2. Ceramic sliding partner according to claim 1, wherein the ceramic material is formed from an oxide-ceramic material.

3. Ceramic sliding partner according to either claim 1, wherein the ceramic material is selected from a mixed oxide system Al2O3-ZrO2, ZTA ceramic materials (zirconia toughened alumina), or ceramic composite materials in which zirconium oxide represents a volume-dominating phase.

4. Ceramic sliding partner according to claim 1, wherein a pore size of a porous region of the ceramic sliding partner is ≥1 nm.

5. Ceramic sliding partner according to claim 1, wherein a porous region of the ceramic sliding partner has a porosity of from 20 to 95%.

6. Ceramic sliding bearing comprising at least one sliding partner A according to claim 1 and at least one sliding partner B, which has a sliding surface, and wherein the sliding surfaces of the at least one sliding partner A and B are configured to be moved against one other.

7. Ceramic sliding bearing according to claim 6, wherein the sliding partner B consists of ceramic material.

8. Ceramic sliding bearing according to claim 6, wherein the sliding partner B consists of solid ceramic material.

9. Ceramic sliding bearing according to claim 6, wherein the sliding partner B is at least partly porous.

10. Use of the ceramic sliding bearing according to claim 6 as implants for human medical or veterinary applications.

11. Use of the ceramic sliding bearing according to claim 10 one or any combination of as an implant for joints, implants in partial resurfacing, and as parts of implant systems.

12. Use of the ceramic sliding bearing according to either claim 10 as an implant for any one of a finger joint, toe joint, elbow joint, ankle joint, wrist, hip joint, knee joint, or shoulder joint.

13. Use of the ceramic sliding bearing according to claim 10 as a partial prosthesis, which compensates only for local joint/cartilage defects.

14. Use of the ceramic sliding bearing according to claim 6 as a technical sliding bearing in a linear, radial, axial and/or radiax bearing.

15. Use of the technical sliding bearing according to claim 14 in turbine wheels.

16. Ceramic sliding partner according to claim 2, wherein the oxide-ceramic material is based on aluminum oxide or zirconium oxide.

17. Ceramic sliding partner according to claim 1, wherein the ceramic material is formed from a non-oxide ceramic material.

18. Ceramic sliding partner according to claim 17, wherein the non-oxide-ceramic material is based on silicon nitride, or silicon carbide.

19. Ceramic sliding partner according to claim 4 wherein the pore size is between 50 μm and 1 mm.
